(19) United States
(12) Patent Application Publication
(10) Pub. No.: US 2026/0234208 A1
(43) Pub. Date: Aug. 13, 2026

(54) METHOD FOR PRODUCING A KERATIN POWDER FROM FEATHERS OF ANIMAL ORIGIN, COLLOIDAL SOLUTION AND KERATIN POWDER FOR TOPICAL APPLICATION CONTAINING KERATIN PARTICLES, AND USE OF THE COLLOIDAL SOLUTION AND THE KERATIN POWDER

(71) Applicant: RIGI THERAPEUTICS AG, Meggen (CH)

(72) Inventor: Christian SPORN, Halle (Saale) (DE)

(73) Assignee: RIGI THERAPEUTICS AG, Meggen (CH)

(21) Appl. No.: 19/153,903

(22) PCT Filed: Feb. 6, 2024
(86) PCT No.: PCT/EP2024/052925

§ 371 Date: Aug. 5, 2025

(30) Foreign Application Priority Data
Feb. 8, 2023 (EP) 23155670.5

(51) Int. Cl. C07K 14/47; A61K 9/00; A61K 9/06; A61K 9/16; A61K 45/06; B82Y 5/00

(52) U.S. Cl. CPC C07K 14/4741; A61K 9/0014; A61K 9/06; A61K 9/1658; A61K 9/1682; A61K 45/06; B82Y 5/00

(57) ABSTRACT

The invention relates to a method for producing keratin powder of the protein β-keratin from feathers of animal origin by extracting keratin, filtering the extraction solution and obtaining a colloidal solution, and drying same to form the keratin powder. The invention also relates to a colloidal solution for topical application containing keratin particles of the protein β-keratin and/or agglomerates thereof or a keratin powder containing said keratin particles. The colloidal solution and the keratin powder are used for the production of preparations with therapeutic, diagnostic, preventative or cosmetic purposes for humans and/or animals.

The present invention relates to a method for producing keratin powder of beta-keratin protein from feathers of animal origin by means of extracting the keratin, filtering the extraction solution to obtain a colloidal solution, and drying it to form keratin powder. The invention also relates to a colloidal solution for topical application comprising keratin particles of the beta-keratin protein and/or agglomerates thereof or a keratin powder comprising these keratin particles. The colloidal solution and the keratin powder are used for the production of preparations with therapeutic, diagnostic, preventive, or cosmetic applications in humans and/or animals.

The stratum corneum (SC) of the epidermis consists of several layers of keratinized corneocytes, which are surrounded by the “cornified envelope” and connected by corneodesmosomal structures and embedded into a complex lipid matrix of liquid crystalline, lamellar structures.

Keratinocyte lipid synthesis is largely autonomously regulated and also provides, in addition to cholesterol and cholesterol derivatives, free fatty acids of different chain lengths as well as triglycerides. In addition, ceramides are synthesized in the endoplasmic reticulum of keratinocytes, said ceramids differing substantially from molecules of other lipid classes due to their anisomeric molecular structure. Due to charge differences within the long-chain molecule, ceramides can spontaneously form lyotropic mesophases, that is, liquid-crystalline membrane structures. In contrast to phospholipids, the functionally important ceramides of the stratum corneum have two alkyl chains, which in turn vary in length. In addition, these have different configurations, depending on the degree of hydration, so that different membrane models describe a complex network of membrane sections with polymorphic phase behavior. The dynamic order of the overall system is increasingly understood in its complexity and is gaining more practical importance in the development of modern galenic vehicle systems.

The barrier function is also substantially determined by the quantity of the hydrophilic phase in microenvironments. The distribution of water within the microcompartments of the stratum corneum may be described in at least two fractions. In addition to a free water phase, a fraction of bound water is defined. The latter consists of a mobilizable subfraction and a fixed subfraction. The nomenclature is thereby aimed at the dynamic exchange of hydrophilic valences between the individual compartments. Fixed water is primarily understood to be the water portion that is bound in corneocytes by strong hygroscopic forces mediated by proteolytically generated amino acids and is virtually available for exchange with a very delayed release. Under special conditions, water bound by swellable membrane parts can be liberated and transferred to the free water phase. This is where the functionally important water phase of the stratum corneum becomes visible. The free water is additionally bound in the individual microcompartments by hygroscopic molecules, which are collectively designated as a “natural moisturizing factor (NMF)”, and is in exchange with the water phase of the vital epidermis as well as the environment (transepidermal water flow). Essential components of the NMF are, in particular, pyrrolidone carboxylic acids, lactate, urea, and inorganic ions, in addition to amino acids. These are synthesized by the keratinocytes and released depending on the degree of differentiation.

In the context of desquamation, corneodesmosomes and proteins of the cornified envelope in the stratum disjunctum, the uppermost layer of the stratum corneum are degraded by proteolysis. However, the predominant keratin of the corneocytes is not directly affected by this. Therefore, the integrity of the corneocytes is maintained for a very long time during desquamation. An essential function of the corneocytes consists in providing water for membrane formation in the intercorneocytic space via diffusion of the cornified envelope. Under therapeutic conditions, the interaction of keratin with epicutaneously applied active ingredients is considered insignificant, since transcellular passage to a relevant degree has not yet been demonstrated for any active ingredient. The cornified envelope, which surrounds the corneocyte as an envelope and barrier membrane, is seen as the cause for this. And yet, the corneocyte indirectly influences the diffusion conditions (diffusion coefficients) of the stratum corneum via its central position as an essential water reservoir of the stratum corneum. The cutaneous bioavailability of epicutaneously applied active substances is primarily determined by the interaction of the galenic system with the stratum corneum as the direct contact layer. From a pharmacological point of view, two processes within the stratum corneum are important for the flow of substances: the barrier function and the reservoir function. It is also thereby important to note that the stratum corneum is not a homogeneous structure, but instead, following formation and compact structuring in the stratum compactum, it is loosened by an enzymatically controlled desquamation process (stratum disjunctum). A diametrical importance within the structural levels of the stratum corneum is thus acknowledged for the two functional areas mentioned. The maximum of the barrier function is accordingly in the stratum compactum and that of the reservoir function in the stratum disjunctum. The latter serves as a privileged acceptor for epicutaneously applied and liberated phases. This is decisive for the overall cutaneous kinetics of an active ingredient, as maximum concentrations in deeper skin layers are reduced and the penetration process is delayed. At the same time, large amounts of liberated active ingredients can be absorbed and become bioavailable even after relatively short application times. Overall, it is clear that the micromorphological conditions, both as anatomical variance (e.g., hairy skin vs. hairless skin), and under pathological conditions (e.g., epidermal proliferation or differentiation disorder), have a direct impact on the pharmacokinetic profile of an epicutaneously applied substance.

This makes it clear that a targeted influence of the water content of the stratum corneum has a direct influence on the physicochemical barrier function, and thus on the pharmacokinetics of epicutaneously applied active ingredients.

The use of keratin for medical or cosmetic indications is known from the following publications.

Keratin available to date is degraded and extracted into smaller peptide structures on the basis of microbial, acid or base-induced hydrolysis, and is no longer present as an intact and complete keratin protein (Shandie, A. et al., Biomater. Sci. 2017, 5, 1699-1735; Gupta, A. et al., J. Chem. Chem. Eng. 2012, 6, 732-737).

These keratin peptides are short-chain in their sequences and have different properties (e.g., molecular size, swelling behavior) than the native, high-sequence keratin protein, which was obtained here for the first time. In contrast to previous processes, the use of hydrolysis for extraction was omitted; instead the protein was made accessible for extraction by breaking the disulfide bridges. By this means, the primary structure and the secondary structure of the keratin protein as a β-pleated sheet were preserved.

Starting from here, it was the object of the present invention to provide colloidal solutions, which function as a basis for preparations that show an interaction with both hydrophilic and also lipophilic components or active ingredients. In addition, these colloidal solutions should be easily producible.

This problem is solved using the method having the features of claim 1, the colloidal solution having the features of claim 7 and the keratin powder having the features of claim 11. Uses according to the invention are specified in claim 13. Further dependent claims list preferred embodiments.

According to the invention, a method is provided for producing keratin particles from feathers of animal origin, in which
a) an extraction of the beta-keratin protein, in particular beta-keratin in its secondary structure as a β-pleated sheet, is carried out in an extraction solution, which triggers chemical denaturation and comprises at least one denaturing agent, at least one base, at least one reducing agent and at least one buffering agent,
b) the extraction solution from step a) is subjected to filtration, in which a colloidal solution of the keratin particles of the beta-keratin protein is obtained,
c) the colloidal solution from step b) is dried by freeze drying, spray drying, vacuum drying, air drying, heat drying, infrared drying, and/or microwave drying to obtain a powder comprising keratin powder.

The present invention thus relates to the use of keratin particles of the beta-keratin protein, which are extracted as keratin protein from bird feathers using chemical denaturation, and are used in liquid or semi-solid preparations for epicutaneous application.

When obtaining keratin from wool hair, alpha and beta-keratin are obtained, so-called “soft fiber” (alpha) or “hard fiber” (beta) keratin, that is, two different types of keratin. The extraction, carried out in this case using urea in the alkaline range, additionally leads to the fact that the native form of beta-keratin is obtained from the feathers as a whole protein, and thus retains a high proportion of its secondary structure, the β-pleated sheet, which determines the high water-binding capacity of this keratin. Beta keratin is obtained, in particular, from bird feathers. It is rich in the amino acids, glycine and alanine, and comprises little cysteine, proline and hydroxyproline.

The interaction of the keratin with hydrophilic substances is thereby used as a reservoir for the targeted influencing of their pharmacokinetics. By combining keratin with water, amino acids, hygroscopic substances, peptides and/or proteins, a substitution of the physical barrier should also be achieved.

The extraction solution preferably has a pH value of 8 to 13, more preferably 9 to 12 and particularly preferably 10 to 11.

It is preferred that the at least one denaturing agent is selected from the group consisting of urea, thiourea, guanidine hydrochloride, sodium dodecyl sulfate (SDS), and mixtures thereof.

It is further preferred that the at least one base is selected from the group consisting of sodium hydroxide, potassium hydroxide, and mixtures thereof.

The at least one reducing agent is preferably selected from the group consisting of β-mercaptoethanol, cysteamines, cysteines, glutathione, sodium disulfite, sodium sulfide, sodium hydrogen sulfite, sodium dithionite, sodium thiosulfate, dithiotreitol (DTT), thioglycolic acid and its salts, thiourea, tris(carboxyethyl)phosphine (TCEP) and other phosphines, ammonium chloride, and mixtures thereof.

It is preferred that the at least one buffering agent is selected from the group consisting of tris(hydroxymethyl)aminomethane, sodium dodecyl sulfate (SDS), Tris/hydrochloric acid (HCl), ethylenediaminetetraacetic acid (Tris-EDTA), potassium chloride-sodium hydroxide (KCl—NaOH), sodium hydrogen carbonate (NaHCO₃), dithiotreitol (Tris-DTT), and mixtures thereof.

It is preferred that the extraction solution comprises at least one of the following chemical components:
at least one oxidizing agent selected from the group consisting of hydrogen peroxide, potassium permanganate, sodium perborate, peroxyacetic acid, performic acid, and mixtures thereof,
at least one acid selected from the group consisting of nitric acid, nitrous acid, hypohalous and hyperhalous acids, and mixtures thereof,
at least one ionic agent selected from the group consisting of 1-butyl-3-methylimidazolium (BMIM) chloride, 1-butyl-3-methylimidazolium (BMIM) bromide, 1-butyl-3-methylimidazolium (BMIM) tetrafluoroborate, amide chloride, and mixtures thereof.

One preferred embodiment of the method according to the invention provides that, during the extraction in step a), at least one of the following steps is carried out:
a mechanical comminution, in particular by grinding via ultrasound, preferably in the frequency range of 20 to 50 hertz, using a ball mill and/or using a cutting mill, preferably Ultra-Turrax, wherein the mechanical comminution is carried out according to sieve analysis to a particle size (d50) of 0.1 to 5.0 mm, preferably 0.2 to 1.0 mm,
a thermal denaturation, in particular at temperatures of 70° C. to 150° C., and/or electrochemical denaturation,
a precipitation of the extraction solution from step a), in particular triggered by a pH change, addition of a co-solvent and/or a salt,
a microbial and enzymatic extraction via
   gram-negative bacteria, selected from the group consisting of Stenotrophomonas sp., Chrysebacterium sp., Vibrio sp., and mixtures thereof,
   gram-positive bacteria, selected from the group consisting of Bacillus sp., Kocuria rosea, and mixtures thereof,
   saprophytic and/or parasitic fungi, and/or
   mixtures thereof,
treatment with microwave radiation, especially microwave radiation up to 960 watts and 2450 hertz,
use of electrical explosion and/or supercritical water, and/or
combinations thereof.

It is preferred that the beta-keratin protein is present in its secondary structure as a β-pleated sheet. In contrast to alpha keratin (=soft fiber), beta-keratin (=hard fiber) has a high proportion of tightly twisted β-pleated sheet structures stabilized by disulfide bridges, which ensure a high stability of the hard fiber keratin.

The filtration is preferably carried out via dialysis and/or ultrafiltration (cross-flow filtration).

The feathers of animal origin are preferably selected from the group consisting of feathers from chickens, geese, ducks, turkeys, pheasants, ostriches, rheas, emus, quails, and mixtures thereof.

A colloidal solution for topical application is likewise provided according to the invention and comprises keratin particles of the beta-keratin protein and/or agglomerates thereof.

It is preferred that the keratin particles in the colloidal solution have a particle size in the range of 5 nm to 500 nm, preferably 70 nm to 350 nm, measured by means of dynamic light scattering (according to DIN ISO 22412:2018-09) (Zetasizer ZEN3600, Malvern Panalytical Instruments).

It is preferred that the colloidal solution comprises additives for stabilization, said additives are preferably selected from the group consisting of
proteins, e.g., albumins,
carbohydrates, such as sucrose, lactose, glucose, fructose, mannitol, sorbitol, and sweeteners, such as saccharin sodium, sodium cyclamate, aspartame, starches and modified starches, cyclodextrins, and/or mixtures thereof,
polyanionic surfactants, such as sodium dodecyl sulfate, sodium cetylstearyl sulfate, cetylstearyl alcohol (emulsifying), sodium dioctyl sulfosuccinate, and/or mixtures thereof,
non-ionic surfactants, such as fatty alcohols and sterols, sorbitan fatty acid esters, polyoxyethylene sorbitan fatty acid esters, polyoxyethylene fatty acid glycerides, macrogol-1000 glycerol mono fatty acid esters, polyoxyethylene fatty acid esters, polyoxyethylene fatty alcohol ethers, glycerol fatty acid esters, sucrose fatty acid esters, poloxamers, and/or mixtures thereof,
gelling agents, such as polyacrylates, cellulose derivatives, such as methylcellulose, methylhydroxypropylcellulose, hydroxypropylcellulose, hydroxyethyl cellulose and/or ethylcellulose, carmellose sodium, and/or mixtures thereof,
thickeners, such as tragacanth gum, xanthan gum, gum arabic, guar galactomannan, alginates, bentonite, and/or mixtures thereof,
film formers such as methacrylic acid acrylates, polyvidone, polyvinyl alcohol, and/or mixtures thereof,
polymers, such as macrogols, gelatin, and/or mixtures thereof.

According to the invention, keratin powders of the beta-keratin protein are also provided for topical application, which may be produced from the colloidal solution described above. The keratin powder is produced by drying the colloidal solution, in particular by freeze drying, spray drying, vacuum drying, air drying, heat drying, infrared drying, and/or microwave drying.

It is preferred that the keratin particles in the keratin powder have a particle size in the range of 1 to 250 μm, preferably 5 to 30 μm, measured by means of dynamic light scattering (according to DIN ISO 22412:2018-09) (Zetasizer ZEN3600, Malvern Panalytical Instruments).

The colloidal solution described above and the keratin powder described above are used for the production of preparations with therapeutic, diagnostic, preventive, or cosmetic applications in living beings, in particular in humans and/or animals.

One preferred embodiment provides that the colloidal solution comprises at least one small-molecule and/or one biotechnological active ingredient. The active ingredient is preferably selected from the group consisting of glucocorticoids, calcineurin inhibitors, Janus kinase inhibitors, antibiotics, and/or a protein in and of itself, a fraction protein, peptide, enzyme, antibody, antibody fragment, RNA and/or DNA molecule, and mixtures thereof. One further preferred embodiment provides that a molecule is included, said molecule representing a target for one of these active ingredients.

It is further preferred that the colloidal solution comprises cosmetic active ingredients and excipients, preferably selected from the group consisting of amino acids, urea, glycerol, hyaluronic acid, sugars and sugar-like derivatives, extracts or waxes from plants, or parts derived from animals, and mixtures thereof.

One preferred embodiment provides that specific and/or non-specific compounds are formed with active substances and/or excipients by covalent and/or non-covalent bonds, preferably via selected groups, consisting of carboxylic acid groups, amino groups, thiol groups, and/or hydroxyl groups.

For stabilizing the colloidal solution with or without active ingredients and/or excipients, these are preferably incorporated as liposomal systems in unilamellar or multilamellar vesicles of different or uniform size by adding amphiphilic molecules. These amphiphilic molecules are preferably selected from the group consisting of:
phospholipids, preferably lecithin, DODAB, DPPC, DSPC, DSTAP, and/or mixtures thereof,
cationic lipids, preferably ALC-0315,
PEG lipids, preferably ALC-0159
prostaglandins and prostaglandin modifications, preferably PGE1, PGD2, PGE2, 15-keto PGE1, and/or mixtures thereof,
ceramides, preferably selected from the group consisting of ceramides of the NS, NH, NP, NDS, AS, AH, ADS, AP head groups with chain lengths C10 to C26, and/or mixtures thereof,
ceramides, preferably selected from the group consisting of ceramides of the EOS, i5 EOH, EOP head groups with chain lengths C₁₀ to C₃₂, and/or mixtures thereof,
cholesterol, cholesterol derivatives, and/or mixtures thereof,
fatty acids, preferably selected from the group consisting of fatty acids with chain lengths C₁₀ to C₃₂, and/or mixtures thereof.

It is preferred that the application on living beings, in particular on humans, relates to the skin, mucous membranes (including conjunctiva) or epidermal appendages (including nails and hair), in particular to substitute for the barrier function or for components of the epidermal barrier.

The colloidal solutions and keratin powders according to the invention are also used for the production of:
liquid bases, in particular solutions, emulsions, suspensions, and/or colloids,
semi-solid bases, in particular suspension ointments, ointments, creams, gels, pastes, colloids, and/or suppositories,
solid bases, in particular powders, tablets, granules, pellets, capsules, and/or inserts.

The colloidal solutions and keratin powders according to the invention are also used for the production of:
food and food supplements,
animal feed,
fertilizers and/or plant protection products for plants and/or soils,
technical additives, e.g., in or as a filter system, adhesive and/or bonding agent, consistency agent, filler, absorber for hydrophilic or lipophilic substances, charged or uncharged substances,
packaging materials and/or consumer goods,
textiles and/or fibers with or without functionality, in particular water-repellent, breathable,
medical products, in particular adhesive plasters, wound dressings, tampons, clothing that supports wound or skin care.

The subject matter according to the invention will be explained in more detail with reference to the following figures and the example, without wanting to restrict said subject matter to the specific embodiments shown here.

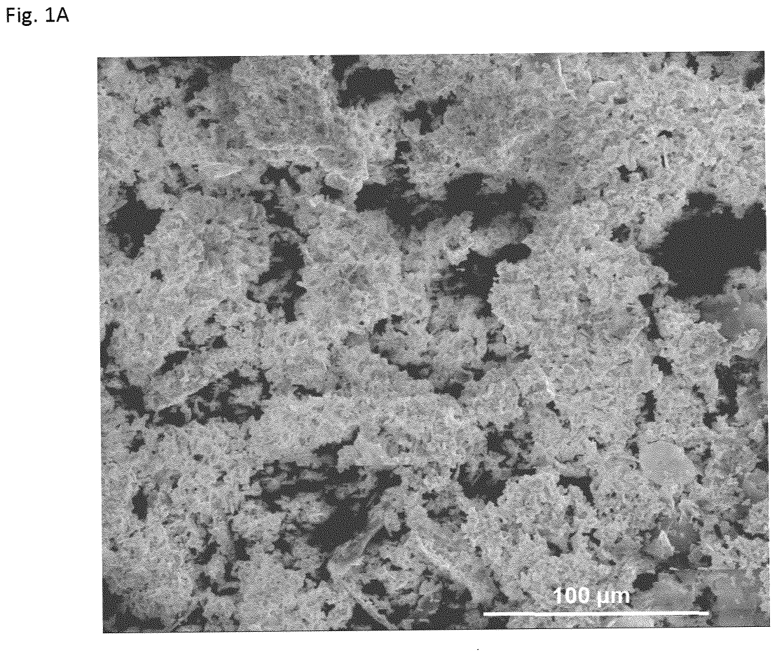

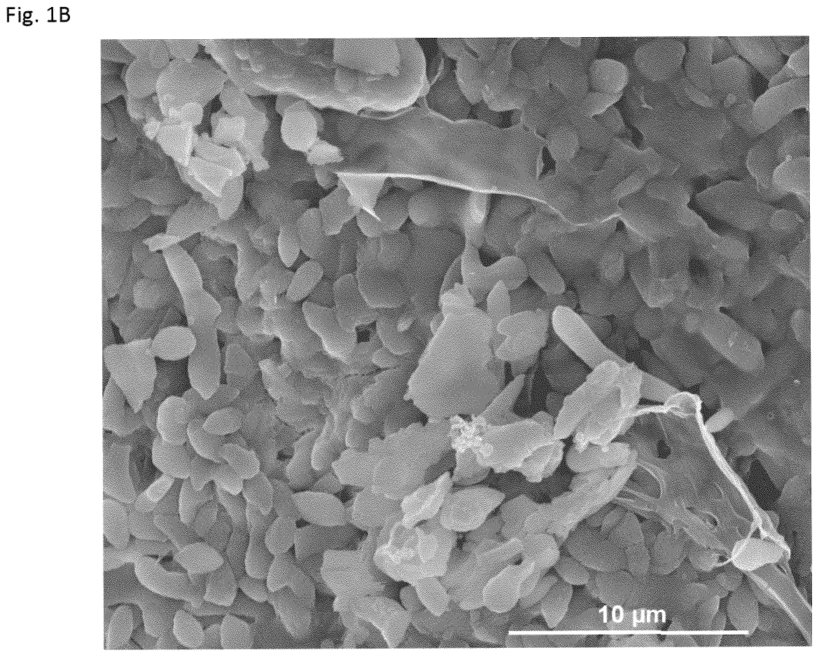

FIGS. 1A and 1B show SEM images of lyophilized hair keratin at different resolutions

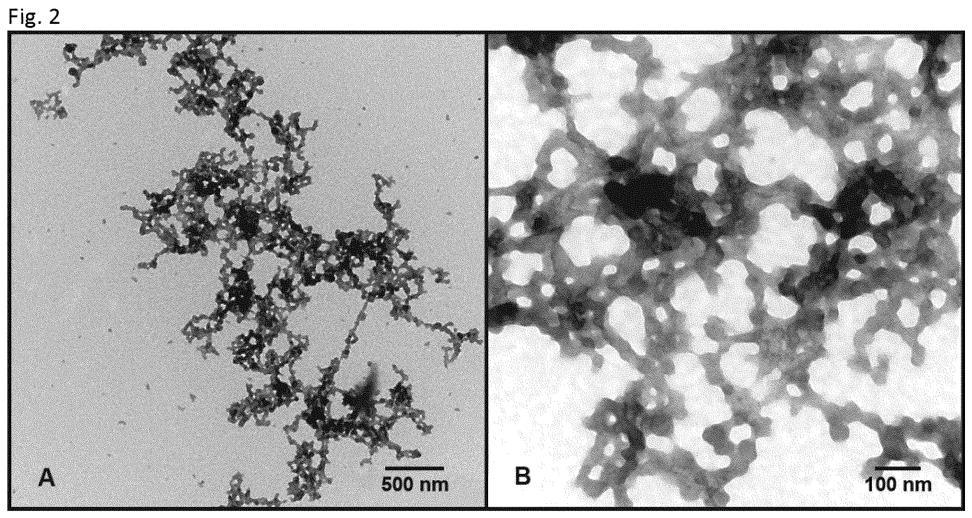

FIG. 2 shows TEM images of A) feather keratin (0.5 mg/ml) and B) hair keratin (0.5 mg/ml)

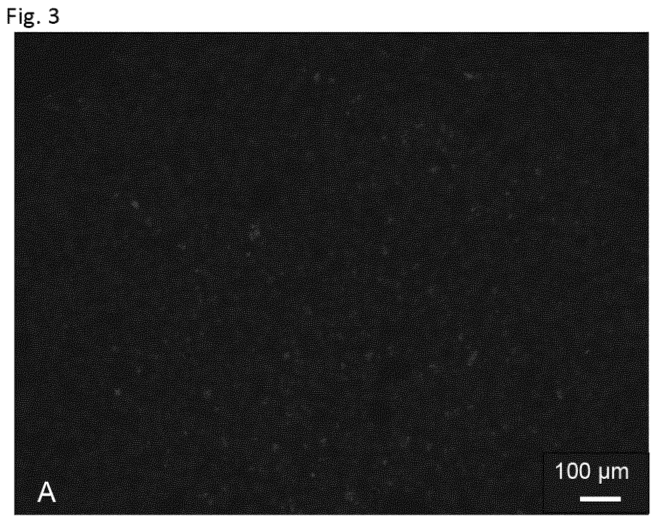

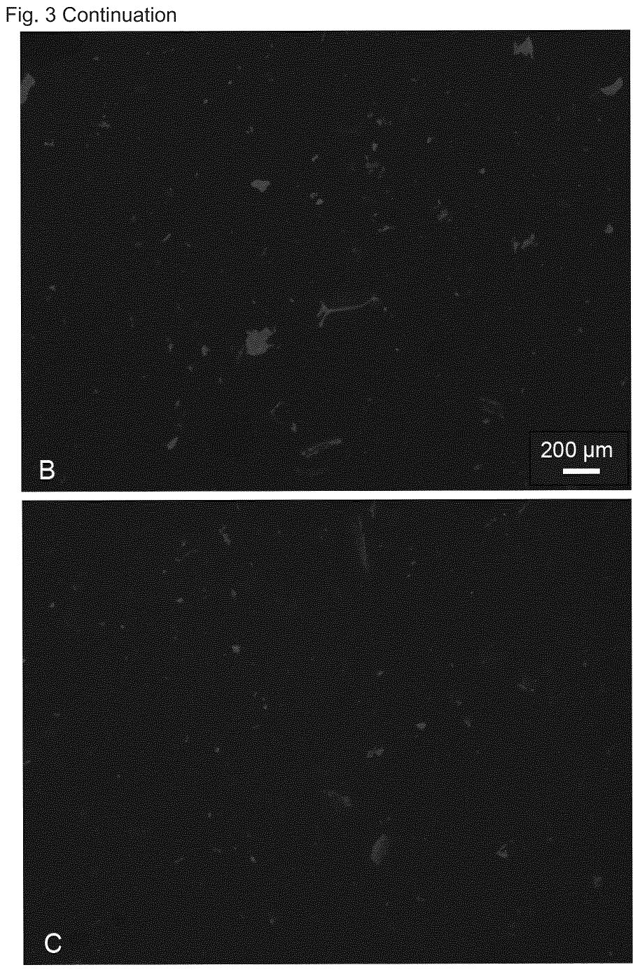

FIG. 3 shows fluorescence spectroscopic images of 5% (w/w) keratin particles in a base cream DAC (with A) feather keratin B) and C) hair keratin)

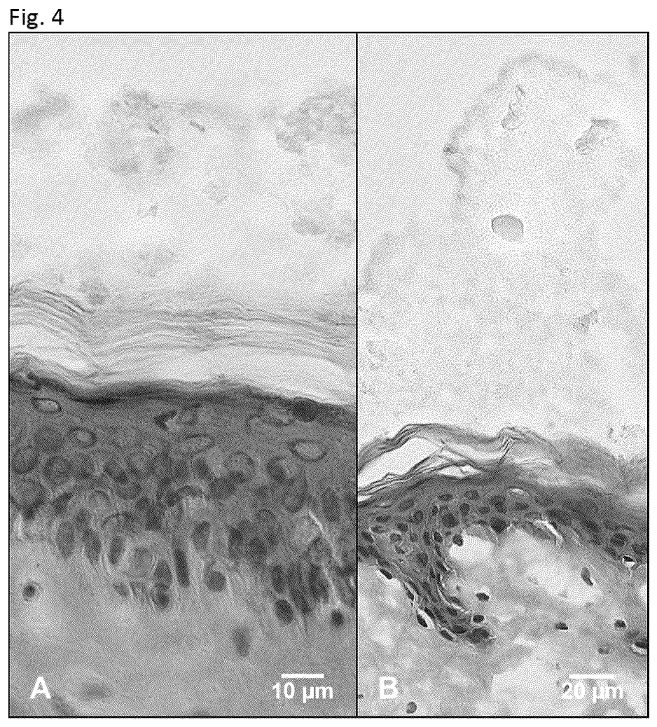

FIG. 4 shows images of penetration tests of A) feather keratin and B) hair keratin with respectively 5% (w/v) in base cream DAC

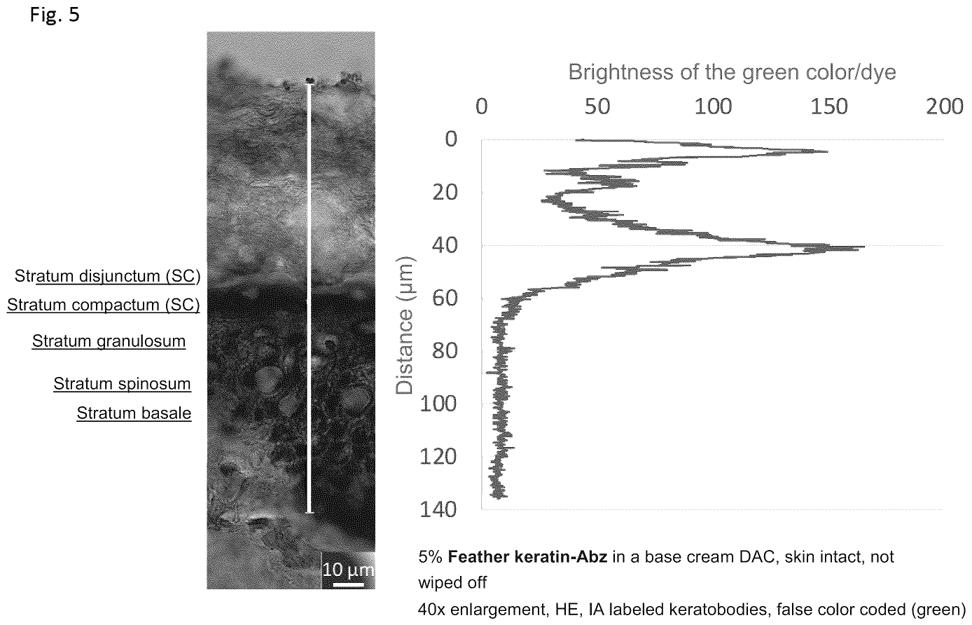

FIG. 5 shows an image of a penetration study of feather keratin 5% (w/v) in a base cream DAC with a fluorescence image

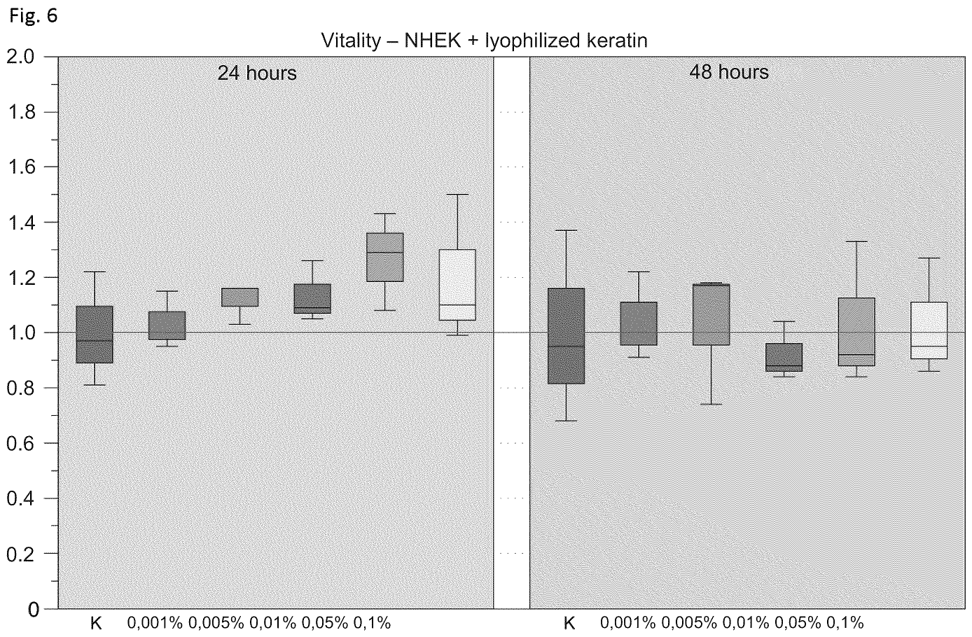

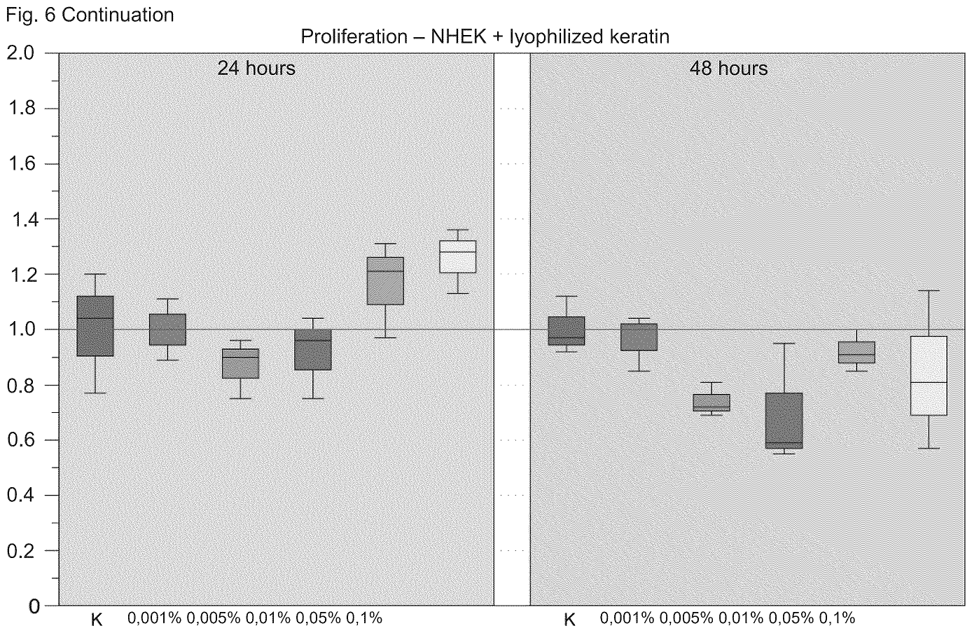

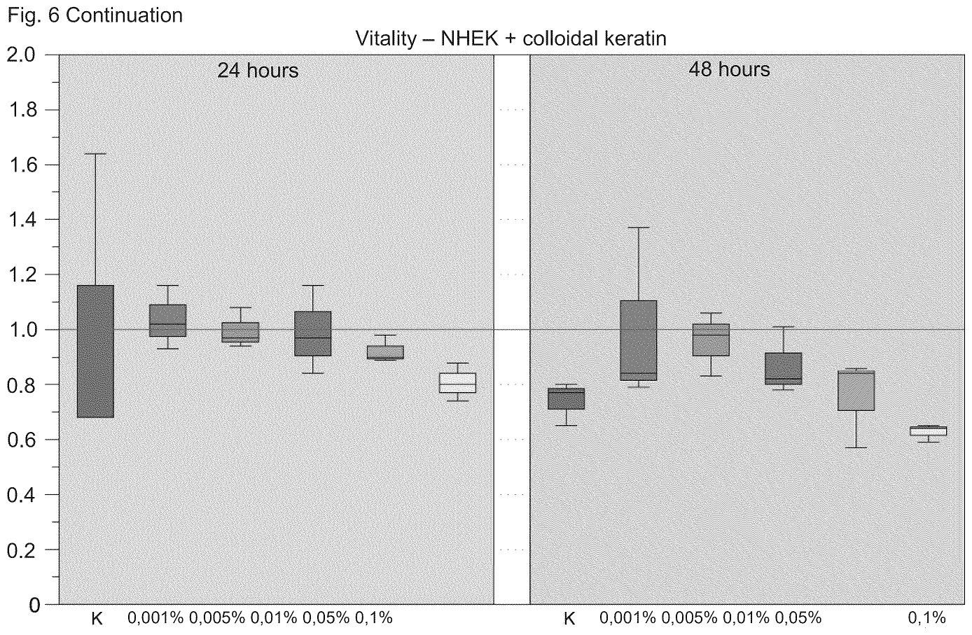

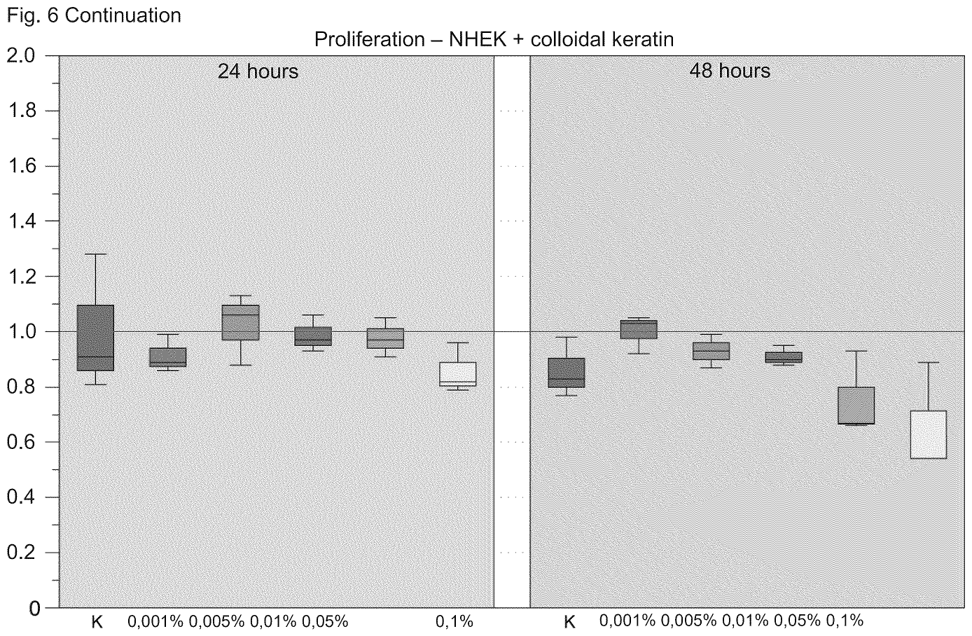

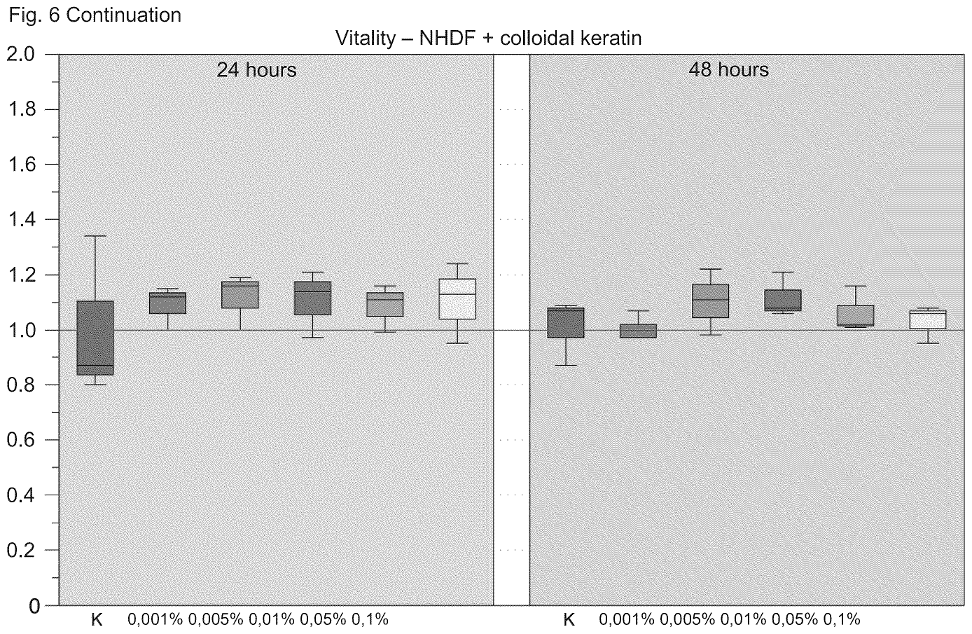

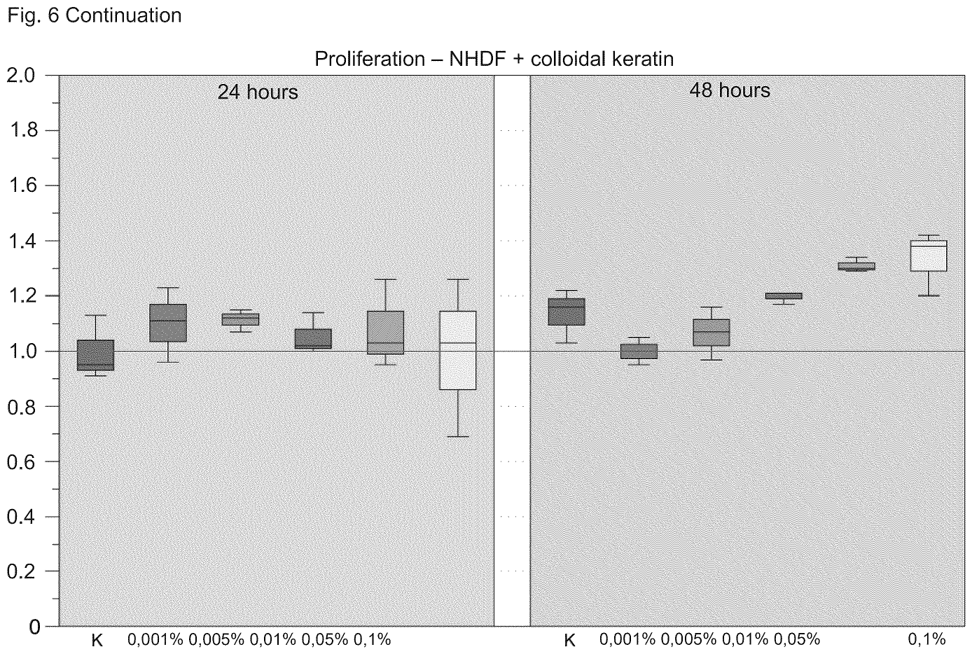

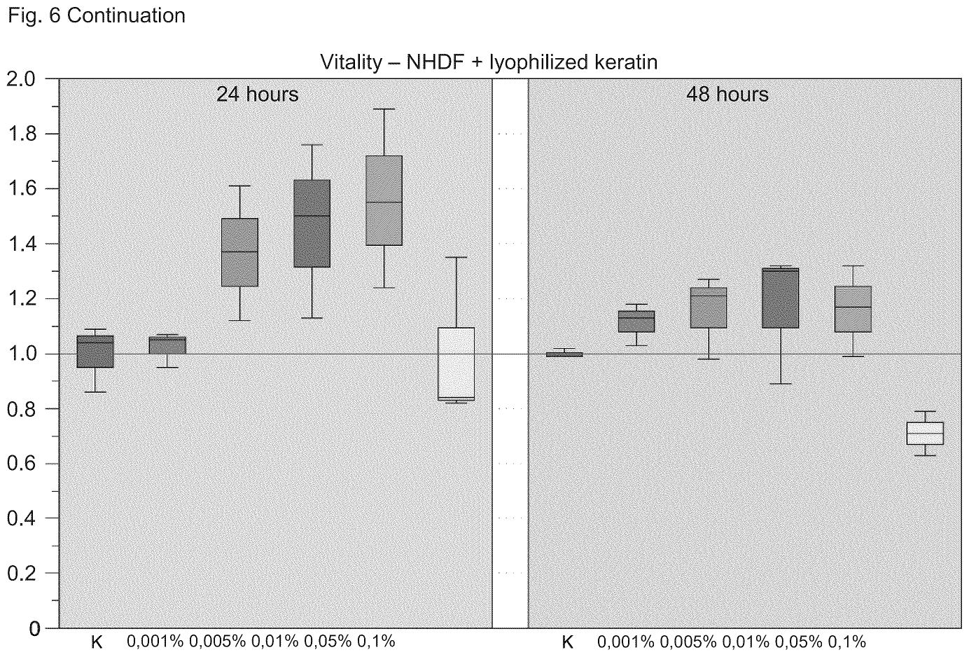

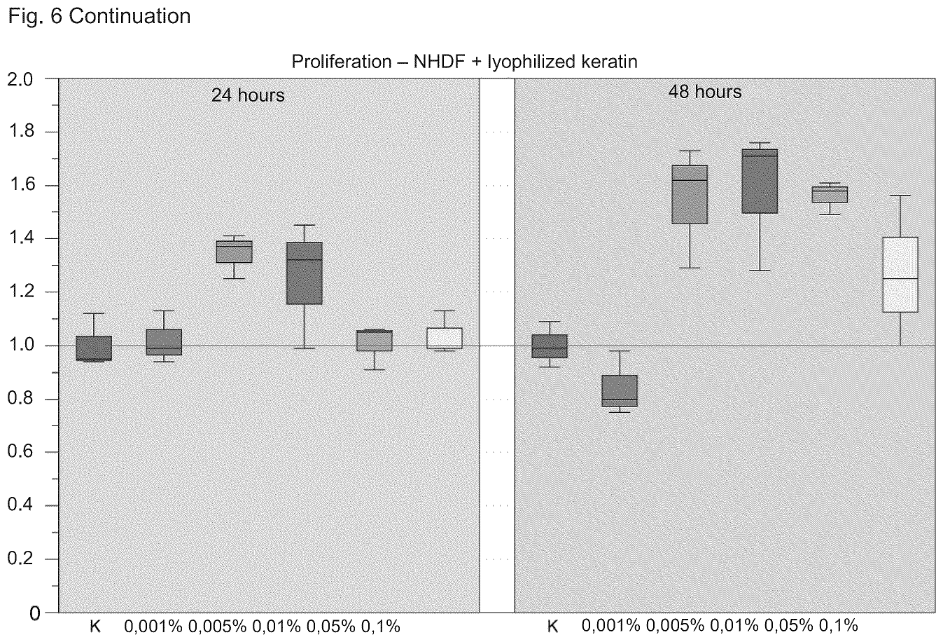

FIG. 6 shows cytotoxicity data as an index of lyophilized and colloidal feather keratin on keratinocytes (NHEK) and lyophilized and colloidal feather keratin on dermal fibroblasts (NHDF)

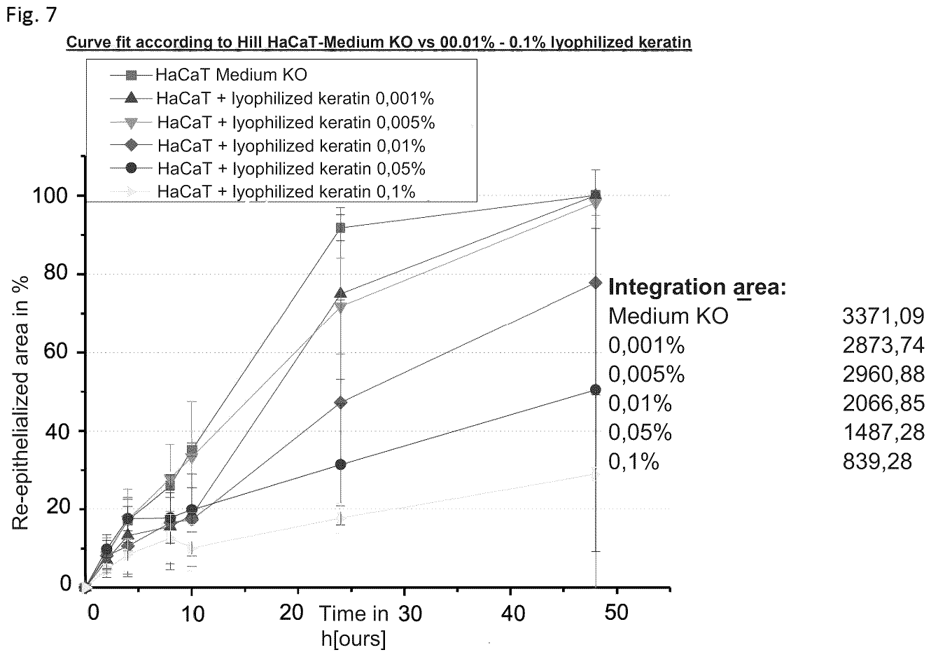

FIG. 7 shows the result of a scratch test depicting the epithelialized area of 3 independent tests on lyophilized feather keratin on keratinocytes (HaCaT)

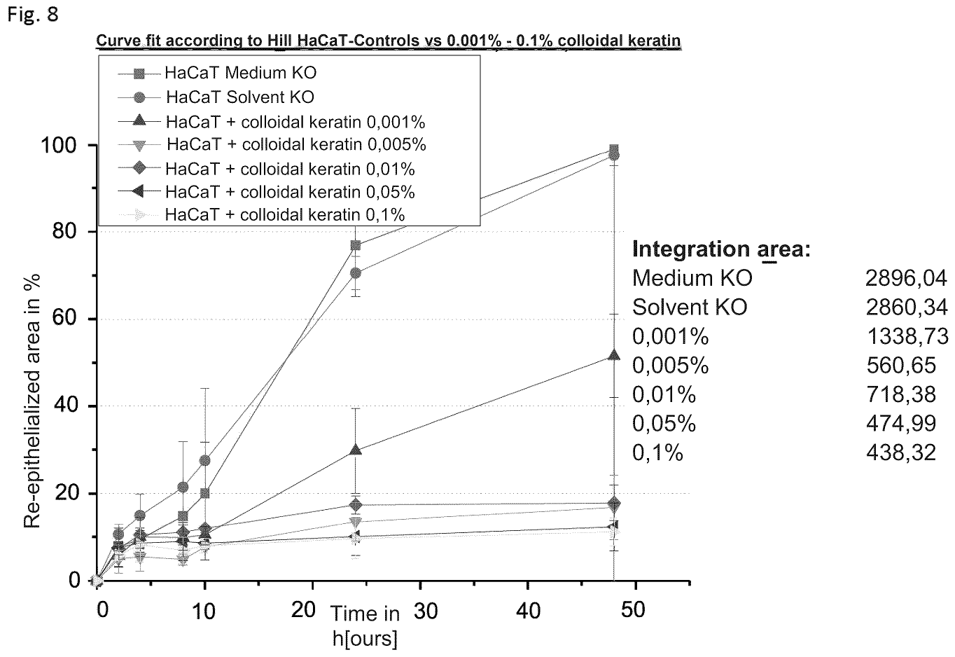

FIG. 8 shows the result of a scratch test depicting the epithelialized area of 3 independent tests on colloidal feather keratin on keratinocytes (HaCaT)

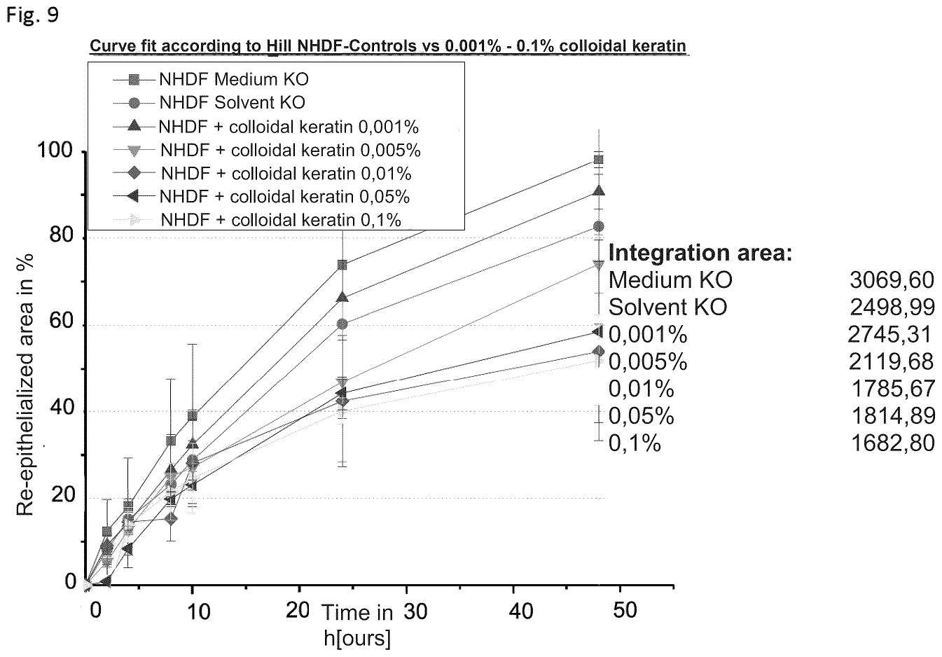

FIG. 9 shows the result of a scratch test depicting the epithelialized area of 3 independent tests on lyophilized feather keratin on keratinocytes (NHDF)

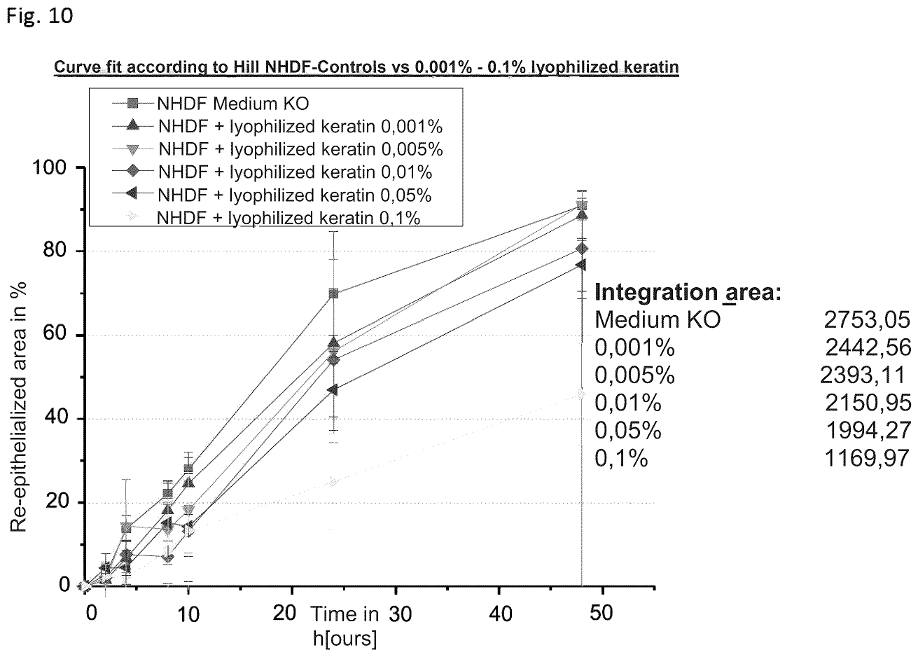

FIG. 10 shows the result of a scratch test depicting the epithelialized area of 3 independent tests on colloidal feather keratin on keratinocytes (NHDF)

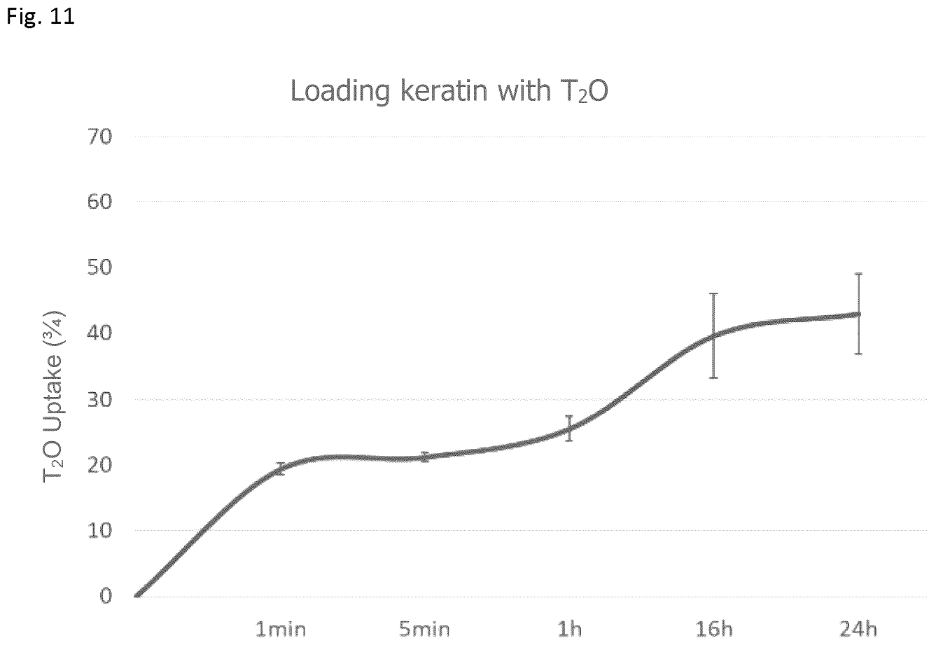

FIG. 11 shows a graph of the water absorption of feather keratin after the addition of 25 μl T₂O (1 μCi) to 5 mg lyophilized keratin, n=3 (contact times: 1 min, 5 min, 1 h, 16 h, 24 h), centrifugation, removal of the supernatant, determination of the radioactive dose

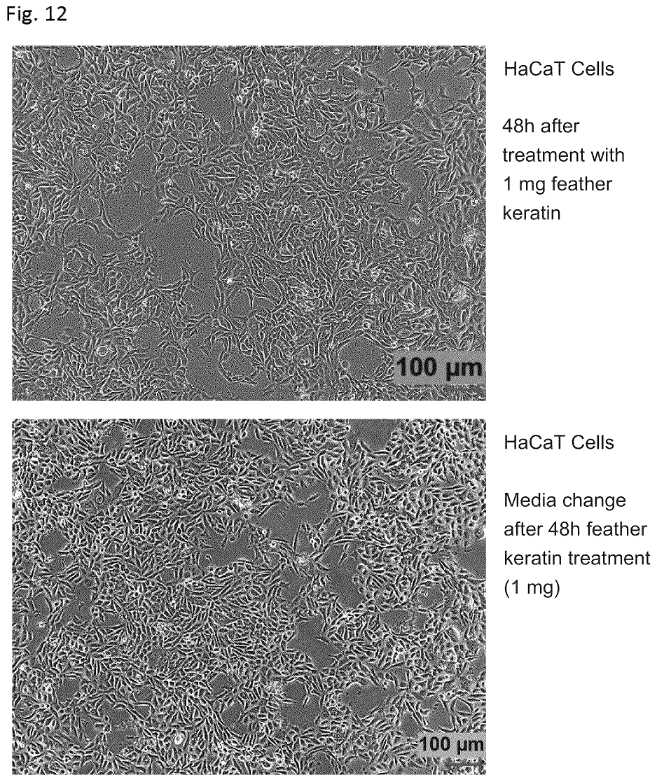

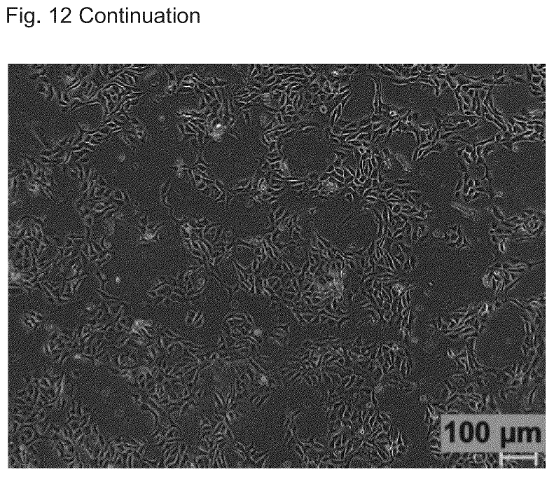

FIG. 12 shows a depiction of the cultivation of HaCaT cells with 1 mg feather keratin

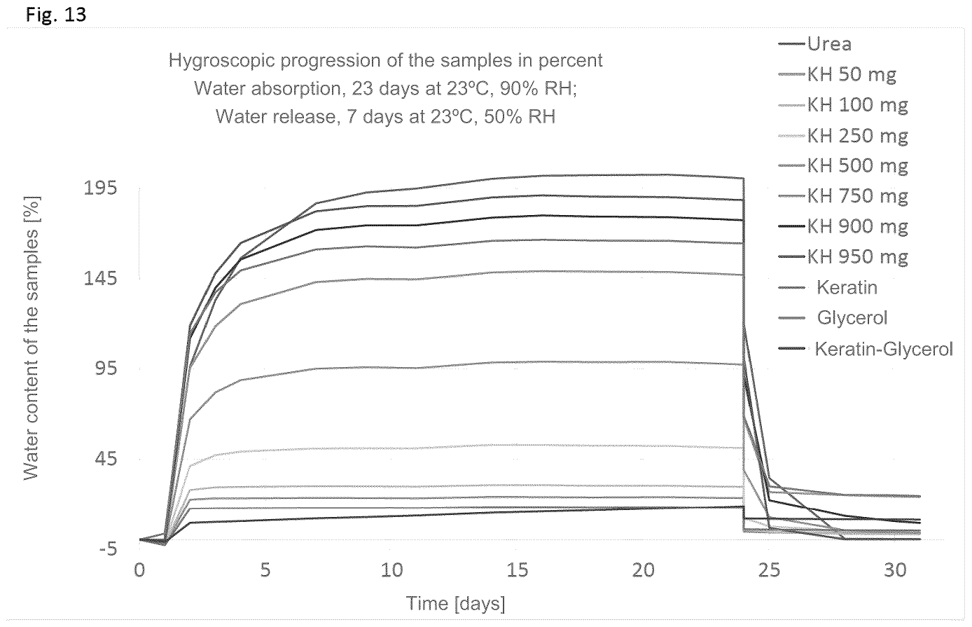

FIG. 13 shows the water absorption of urea, glycerol, and keratin, as well as mixtures of keratin particles with glycerol (50:50) and keratin particles with urea in different ratios (e.g., 95:5 (orange); 50:50 (light blue); 5:95 (brown)) in a climate chamber (room temperature and 95% relative humidity) over time

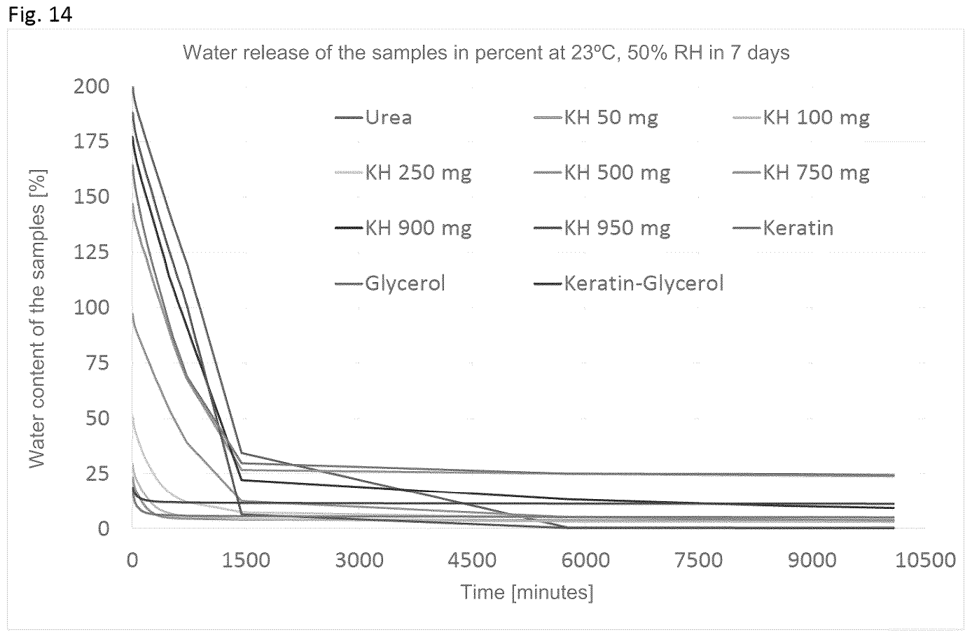

FIG. 14 shows the water release of urea, glycerol and keratin, as well as mixtures of keratin particles with glycerol (50:50) and keratin particles with urea in different ratios (e.g., 95:5 (orange); 50:50 (light blue); 5:95 (brown)) in the climate chamber (room temperature and 50% relative humidity) over time

FIG. 15 shows the tertiary structure of beta-keratin from bird feathers

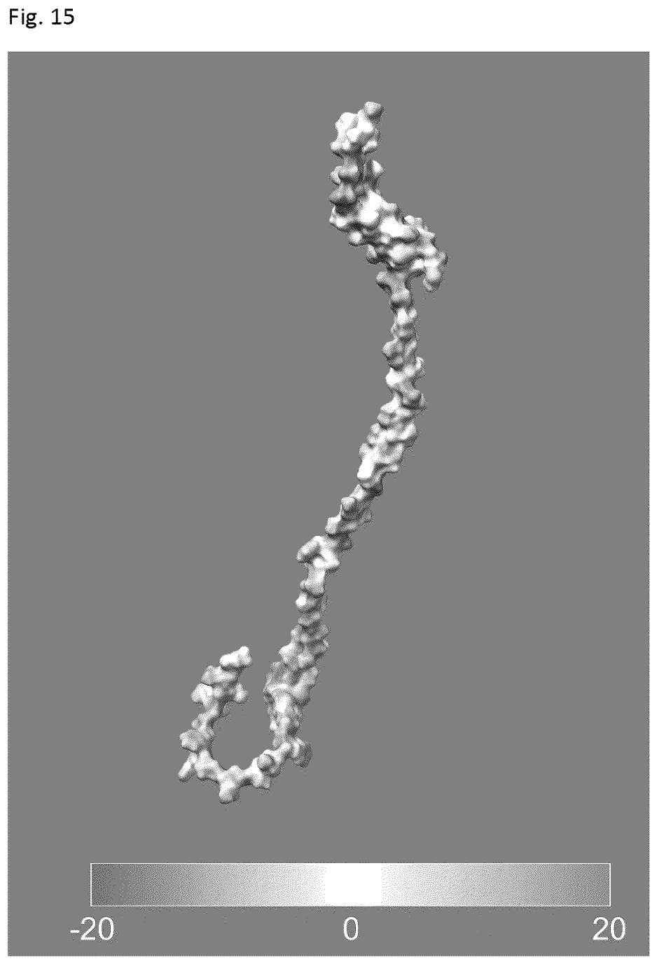

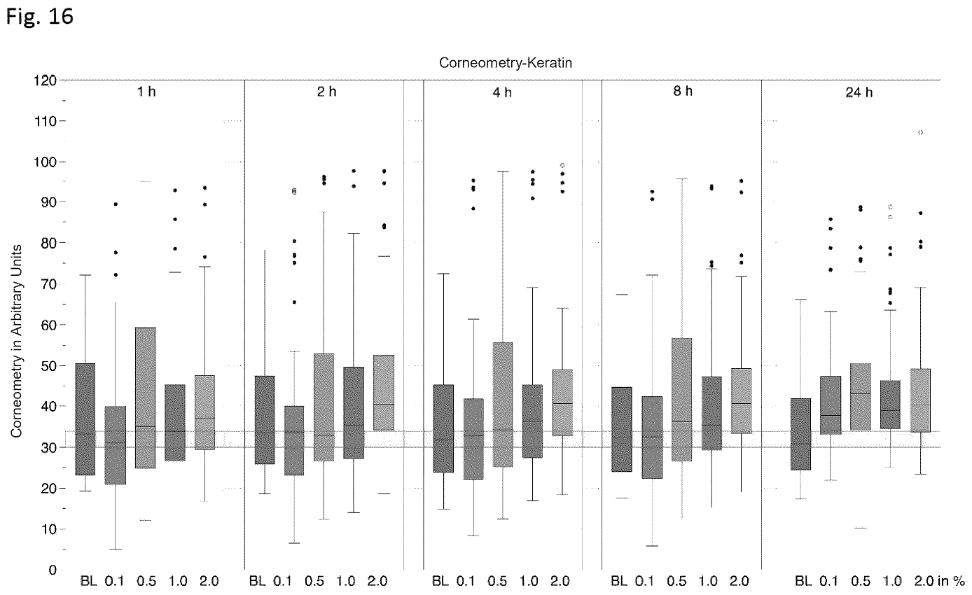

FIG. 16 shows the measurement data of the corneometry in arbitrary units (AU) before and after 1, 2, 4, 8 and 24 hours for a cosmetic cream formulation with 0.1%, 0.5%, 1.0% and 2.0% keratin particles

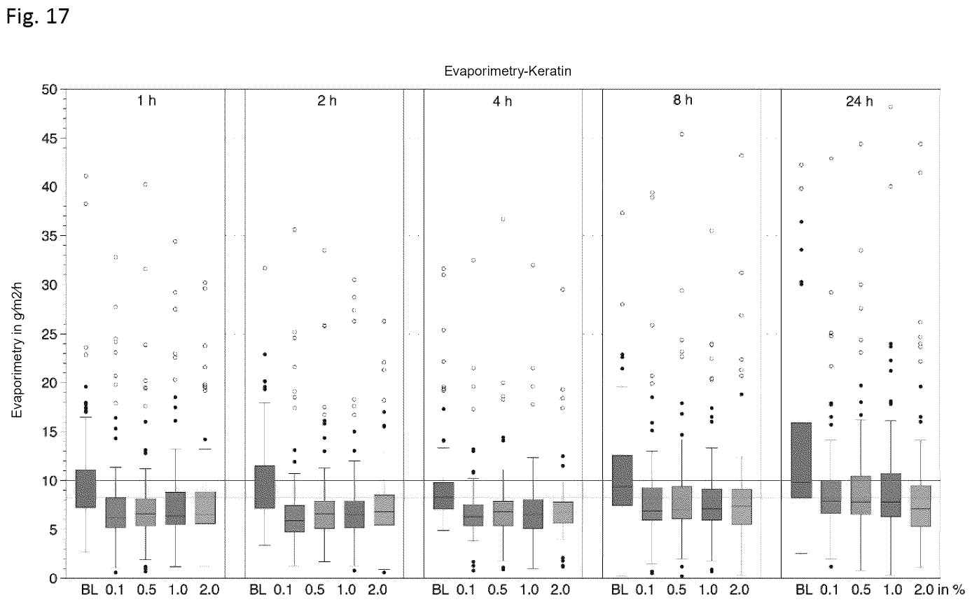

FIG. 17 shows the measurement data of the evaporimetry in g/m²/h before and after 1, 2, 4, 8 and 24 hours for a cosmetic cream formulation with 0.1%, 0.5%, 1.0% and 2.0% keratin particles

Various animal materials are available as a source of keratin; however, they appear to be of varying suitability due to their degree of protein cross-linking. After preliminary investigations using various keratin-comprising biomaterials, bird feathers were shown to be particularly suitable. In addition to practical aspects of availability and ease of processing, biochemical aspects, in particular, were also developed that justify a preferential use of feathers. The keratin contained in feathers corresponds predominantly to β-keratin, which is provided as a polypeptide chain with a β-pleated sheet structure, consists of filaments of 3-4 nm, and has a molecular mass of approximately 10-22 kDa. In contrast to α-keratin in mammals, β-keratin has only a few, yet functionally significant, differences in the primary sequence. Thus, β-keratin forms fewer macrofibrils than α-keratin and shows a more regular ordering and packing behavior. These differences thus offer more favorable prerequisites for a standardized keratin isolation and for the product properties resulting therefrom. In particular, the commonly-practiced hydrolysis of keratin in order to isolate intact keratin proteins is thereby omitted, and the formation of keratin fragments, amino acids and peptides is specifically avoided. For an effective and standardized keratin isolation, a separate process was developed based on the biochemical characteristics of β-keratin. The chicken feathers used were first cleaned with water and soap, disinfected with 70% ethanol, and then dried at room temperature. After comminuting the cleaned and dried feathers in a cutting mill (Retsch SM 100 comfort), a homogenization of entire material was carried out. The feather homogenate was defatted by means of a Soxhlet apparatus. To extract the keratin, the defatted feather material was added to an extraction buffer and extracted for 48 hours. The extract was subsequently centrifuged and the sediment discarded. The pure extract obtained was diluted with water and subjected to filtration with a cut off of 10,000 NMWC. The dialysate was processed into a powder with a particle size of <25 μm by means of spray drying.

To determine the colloidal size of the keratin particles (1 mg/ml deionized water), measurements were carried out using dynamic light scattering (DLS) using the Zetasizer ZEN3600 device from Malvern Panalytical Instruments in accordance with DIN ISO 22412:2018-09. The analytical method enables the characterization of particle sizes in suspensions and emulsions by detecting the emitted scattered light of a laser. The goal was to determine the particle size of the protein in the colloidal state. To measure the DLS, 1 ml of a 0.1 mM colloidal keratin solution was pipetted into a disposable polystyrene cuvette and subsequently transferred to the cuvette module of the Zetasizer Lab, which was heated to 25° C. The particle size was determined using the automatic analysis mode (general purpose) and backward light scattering at an angle of 173°. Each measurement was carried out in triplicate and comprised 15 measurement cycles per run. The measurement data recorded per sample were then averaged. A total of three samples were examined using DLS. Within the context of the DLS, the polydispersity index (PdI), which indicates how homogeneously the particles are distributed within the sample, the size of the particles (in nm) and the percentage of the respective particle size in relation to the total sample content, were determined.

To determine the molecular size (molecular weight) of the extracted keratin particles, a sodium dodecyl sulfate polyacrylamide gel electrophoresis (SDS-PAGE) was carried out in an SDS gel chamber (Mini Gel Tank from Invitrogen). The samples were denatured according to the manufacturer's protocol for NuPAGE® Bis-Tris Mini Gel Electrophoresis (Thermo Fisher). For this purpose, 2 μl sample, 2.5 μl NuPAGE® LSD sample buffer (4×), 1 μl NuPAGE® LSD reducing agent (10×), and 6.5 μl DI water were combined and incubated for 10 min at 95° C. To carry out the gel electrophoresis, the gel chamber was filled with a 1×Tris-Tricine running buffer (1.2 M Tris, 0.8 M Tricine, 2% SDS). The Tris-Tricine gel (Novex 10-20% Tricine gels from Invitrogen (Thermo Fisher Scientific), LOT 20101945, REF EC6625BOX) was subsequently inserted and the comb was removed from the gel. In each case, 10 μl of the prepared samples and 2 μl of the marker were added to the corresponding gel wells. The gel electrophoresis was carried out at 130 V and 250 mA for 1.5 hours. The gel was subsequently removed from the chamber and the running buffer was discarded. The plastic gel holder was broken open and the Tris-Tricine gel was transferred to a container with Coomassie Brilliant Blue G250 stain (2 ml 5% Coomassie solution, 3 ml orthophosphate acid, 20 ml ethanol, 10 g ammonium sulfate, 65 ml dH2O). The gel was incubated in the solution overnight on the shaker. The staining solution was subsequently removed. The gel was rinsed several times with distilled water while shaking gently for discoloration.

### EXAMPLE

An attempt was made to recreate the functionality of a corneocyte. For this purpose, keratin was extracted from various natural keratin sources and corresponding particles were generated, which were initially studied as a mechanical stabilization of lipid matrices in various semi-solid preparations. The intention thereby was to coat the keratin particles with bipolar lipids in order to develop keratosomes.

First, various extraction methods were tested on human hair (alpha-keratin). Chemical denaturation was predominantly used for the decomposition of the hair. Urea, thiourea, and guanidinium hydrochloride were used for this. To support the denaturation, β-mercaptoethanol, cysteamine and L-cysteine were used as reducing agents. The highest yields were achieved with an extraction solution of 5 M guanidinium hydrochloride, 10% cysteamine and 25 mM Tris at pH 8.5. Due to the safety of the substances, urea and L-cysteine, an extraction solution consisting of 10 M urea, 100 mM L-cysteine, 25 mM Tris-HCl at pH 10.5 was selected for isolating the keratin. After the successful extraction, the extraction solution was dialyzed (cutoff: 6-8 kDa, regenerated cellulose, SpectraPor®), a colloidal solution was thereby produced: keratin precipitated out in the dialysis tube at high protein concentrations and large dialysis steps (5-6 I). Finally, the dialysate was lyophilized, which produced a white powder. This powder was examined using scanning electron microscopy (SEM) (FIG. 1). The particles from hair keratin vary in size and shape. It was thereby defined that the particles in the final formulation were to have a size of >600 nm to avoid penetration through the stratum corneum of the skin.

For regulatory reasons, feather keratin (beta-keratin) from various bird species was used as an alternative keratin source. Raw feathers from chickens, geese and ducks were examined for this purpose. For practical reasons, chicken feathers, in particular, were used for further examinations.

Therefore, the extraction of feather keratin was adapted to the established extraction process for hair keratin. It was observed that, in comparison to all other extraction methods examined, the highest yield was achieved using the selected extraction solution consisting of 10 M urea, 100 mM L-cysteine, 25 mM Tris-HCl at pH 10.5. The optional addition of 1 M ammonium chloride prevents carbamylation of the protein and the associated changes in the protein properties.

To determine sizes, the feather and hair keratin particles were comparatively examined using transmission electron microscopy (TEM) in negative stain (FIG. 2). Hair keratin particles in the colloidal solution had a size of approximately 40-75 nm, whereas the feather keratin particles had a size of approximately 20-35 nm. To visualize the keratin particles for the penetration study, they were fluorescently labeled using 2-aminobenzoyl (Abz) (reaction with isatoic anhydride under basic, denaturing conditions). 5% (w/w) fluorescently labeled hair and feather keratin were formulated into a base cream DAC and examined using a fluorescence microscope after smearing on a microscope slide (FIG. 3). The individual particles from the colloidal solutions, which were identified using TEM, form proportional aggregates in a base cream DAC. A homogeneous distribution of the aggregated particles can be seen for the feather keratin, with particle sizes ranging between 20 and 35 μm. Hair keratin, in contrast, aggregates into larger, crystal-like particles. The particle sizes here are between 20 and 140 μm.

Using penetration tests of hair and feather keratin (5% (w/w) in each case) on ex vivo human skin (Franzzelle) in base cream DAC, it could also be additionally demonstrated that the keratin particles do not diffuse into the deeper layers of the skin, but remain in the upper portions of the SC (stratum disjunctum) (FIG. 4 and FIG. 5). In order to better assess the interaction of the feather keratin particles with the SC lipids or the effects of lipid coatings, the zeta potential was determined at different pH values (Table 1).

TABLE 1
Zeta potential of feather keratin and fluorescently labeled feather
keratin, in each case, 10 mg/ml in 10 mM potassium chloride
at different pH values. Measurement carried out by Fabio.

| pH value | Feather keratin, 10 mg/mL | | Feather keratin with Abz |
|---|---|---|---|
| 10.5 | −25 | mV | −24.2 mV |
| 7.6 | −1.5 | mV | −23.3 mV |
| | Experiment 1: −17.8 mV | | |
| 5.5 | −20.2 | mV | −23.3 mV |

The keratin particles have a negative charge, which makes them ideal for coating with positively charged lipids.

Cytotoxicity studies were carried out using standard protocols regarding the influence of colloidal and lyophilized keratin particles on the vitality and proliferative activity of keratinocytes and dermal fibroblasts. No relevant effect on dermal fibroblasts was found at up to 48 hours of incubation time (FIG. 6). In keratinocytes, only colloidal keratin showed a slight, concentration-dependent reduction in proliferative activity.

The influence of the keratin particles in colloidal and lyophilized form on the migration behavior of keratinocytes (HaCaT) and dermal fibroblasts (NHDF) was examined using a scratch assay (FIGS. 7 to 10). This revealed an obvious mechanically-caused inhibition of migration depending on the concentration of the colloidal and, to a much lesser extent, the lyophilized keratin particles. However, no evidence of toxic effects was found.

Loading and deloading tests were carried out in order to objectively determine the loading capacity of the feather keratin particles for hydrophilic or hydrophobic substances. First, the keratin particles were incubated in T₂O solution for different lengths of time and the amount of tritium absorbed was subsequently measured by means of centrifugation (FIG. 11). Only 25-32% of the introduced T₂O could be recovered in the pellet. This showed an unexpectedly high level of interaction between both hydrophilic substances and the extracted feather keratin.

In order to examine possible interactions of the keratin particles with vital cells, HaCaT cells (keratinocytes) were cultured with fluorescently labeled feather and hair keratin particles. The medium was changed 48 hours after the keratin treatment. The keratin particles are thereby clearly visible under fluorescence microscopy and show an association in the vicinity of the cell nucleus, so that an absorption of the particles into the cell could not be ruled out using fluorescence microscopy (FIG. 12). Therefore, the adhesion zone of the keratin particles with the cell membrane was examined using Raman spectroscopy, but no evidence of intracytoplasmic keratin particles could be found. Thus, due to the zeta potential and the charge of the phospholipids of the cell membrane, an adherence of the keratin particles to cell membranes is to be assumed.

In addition, hygroscopic examinations were carried out to determine the water absorption capacity of the keratin particles, in accordance with the functioning of corneocytes. The keratin particles thereby showed a maximum water absorption of around 20% after just 3 days. When urea was added, up to a ratio of 5% keratin to 95% urea, the maximum was reached increasingly later, in the maximum case only after 15 days. In this case, the water absorption capacity was 197%. (FIG. 13). The water release was determined at room temperature and a humidity of 50%. Pure keratin released some water slowly, and still contained water even after 7 days, while for urea, a rapid water release of around 170% was observed within the first 24 hours, up to a complete water release after 4 days. For the mixtures of keratin and urea in the ratios 25:75 and 10:90 and 5:90, a water content of 25% was still observed even after 4 days (FIG. 14).

According to the known keratin sequence and the amino acid classification according to Dan Cojocari, keratin comprises only around 30% hydrophobic and approximately the same number of hydrophilic amino acids, but no binding pockets. Therefore, the demonstrated interaction of keratin with substances, which have different physicochemical properties, is not predictable. Therefore, the tertiary structure of keratin was calculated and the hydrophobic and hydrophilic regions in the molecule were identified by means of the artificial intelligence (AI) AlphaFold 2 (EMBL's European Bioinformatics Institute (EMBL-EBI), Hinxton, UK) (FIG. 15). Here too, the assumption of the absence of binding pockets was confirmed, so that the observed interactions can only be explained on the basis of hydrophobic or hydrophilic interactions between amino acid clusters on the protein side with relevant groups of other molecules.

To demonstrate clinical efficacy, a cosmetic cream formulation comprising keratin particles in increasing concentrations (0.1%, 0.5%, 1.0% and 2.0%) was examined with regard to its hydrating effect on the stratum corneum of healthy volunteers in a prospective, randomized, and double-blind study. The total water content in the stratum corneum (corneometry) and the free water phase as transepidermal water loss (TEWL) was determined by means of evaporimetry after a single application test before (baseline) and after 1, 2, 4, 8, and 24 hours. After 24 hours, there was an approximately 30% increase in the water content of the stratum (FIG. 16) and an approximately 40% reduction in the transcorneal water loss (FIG. 17). These results demonstrate that the extracted keratin particles, as a colloidal aqueous phase in a cream formulation, impart a lasting hydrating effect to the stratum corneum in accordance with the physiological corneocyte function.

1-16. (canceled)

17. A method for producing keratin particles from feathers of animal origin, in which
(a) an extraction of the beta-keratin protein is carried out in an extraction solution, which triggers chemical denaturation and comprises at least one denaturing agent, at least one base, at least one reducing agent, and at least one buffering agent,
(b) the extraction solution from step a) is subjected to filtration, in which a colloidal solution of keratin particles of the beta-keratin protein is obtained, and
(c) the colloidal solution of keratin particles from step b) is dried by freeze drying, spray drying, vacuum drying, air drying, heat drying, infrared drying, and/or microwave drying to obtain a keratin powder.

18. The method according to claim 17, wherein the extraction solution has a pH value of 8 to 13.

19. The method according to claim 17, wherein the at least one denaturing agent is selected from the group consisting of urea, thiourea, guanidine hydrochloride, and sodium dodecyl sulfate (SDS),
and/or
the at least one base is selected from the group consisting of sodium hydroxide and potassium hydroxide,
and/or
the at least one reducing agent is selected from the group consisting of β-mercaptoethanol, cysteamines, cysteines, glutathione, sodium disulfite, sodium sulfide, sodium hydrogen sulfite, sodium dithionite, sodium thiosulfate, dithiothreitol (DTT), thioglycolic acid and its salts, thiourea, tris(carboxyethyl)phosphine (TCEP) and other phosphines, and ammonium chloride,
and/or
the at least one buffering agent is selected from the group consisting of tris(hydroxymethyl)aminomethane, sodium dodecyl sulfate (SDS), Tris/hydrochloric acid (HCl), ethylenediaminetetraacetic acid (Tris-EDTA), potassium chloride-sodium hydroxide (KCl—NaOH), sodium hydrogen carbonate (NaHCO₃), and dithiotreitol (Tris-DTT).

20. The method according to claim 17, wherein the extraction solution comprises at least one oxidizing agent selected from the group consisting of hydrogen peroxide, potassium permanganate, sodium perborate, peroxyacetic acid, and performic acid,
and/or
the extraction solution comprises at least one acid selected from the group consisting of nitric acid, nitrous acid, hypohalous acid, and hyperhalous acid,
and/or
the extraction solution comprises at least one ionic agent selected from the group consisting of 1-butyl-3-methylimidazolium (BMIM) chloride, 1-butyl-3-methylimidazolium (BMIM) bromide, 1-butyl-3-methylimidazolium (BMIM) tetrafluoroborate, and amide chloride.

21. The method according to claim 17, wherein, during the extraction in step a), at least one of the following steps is carried out:
(a) mechanical comminution by grinding via ultrasound in a frequency range of 20 to 50 hertz or by utilizing a ball mill and/or a cutting mill, wherein the mechanical comminution is carried out to a particle size of 0.1 to 5.0 mm,
(b) thermal denaturation and/or electrochemical denaturation,
(c) precipitation of the extraction solution from step a) which is triggered by a pH change, addition of a co-solvent, and/or a salt,
(d) microbial and enzymatic extraction via
gram-negative bacteria selected from the group consisting of Stenotrophomonas sp., Chrysebacterium sp., Vibrio sp., and mixtures thereof,
gram-positive bacteria, selected from the group consisting of Bacillus sp., Kocuria rosea, and mixtures thereof,
saprophytic and/or parasitic fungi, and/or
a combination thereof,
(e) treatment with microwave radiation, and
(f) electrical explosion and/or supercritical water.

22. The method according to claim 17, wherein the filtration is carried out via dialysis and/or ultrafiltration.

23. A colloidal preparation suitable for topical application comprising keratin particles of the beta-keratin protein.

24. The colloidal preparation of claim 23, wherein the beta-keratin is in its secondary structure as a β-pleated sheet, and/or agglomerates thereof obtained from feathers of animal origin.

25. The colloidal preparation according to claim 23, wherein the keratin particles have a particle size in the range of 5 nm to 500 nm, as measured by means of dynamic light scattering.

26. The colloidal preparation according to claim 24, wherein the feathers of animal origin are selected from the group consisting of feathers from chickens, geese, ducks, turkeys, pheasants, ostriches, rheas, emus, quails, and mixtures thereof.

27. The colloidal preparation according to claim 23, which comprises additives for stabilization.

28. The colloidal preparation according to claim 27, wherein the additives are selected from the group consisting of
proteins,
carbohydrates,
polyanionic surfactants,
non-ionic surfactants,
gelling agents,
thickeners,
film formers, and
polymers, and
mixtures thereof.

29. A keratin powder suitable for topical application produced from the colloidal preparation according to claim 23.

30. The keratin powder according to claim 29, wherein the keratin particles in the keratin powder have a particle size in the range of 1 to 250 μm, as measured by dynamic light scattering.

31. A method of treating a human and/or animal for a condition relating to skin, mucous membrane, or epidermal appendage of the human and/or animal, the method comprising applying to the human and/or animal the colloidal preparation according to claim 23.

32. A method of treating a human and/or animal for a condition relating to skin, mucous membrane, or epidermal appendage of the human and/or animal, the method comprising applying to the human and/or animal the keratin powder according to claim 29.

33. The method according to claim 31, wherein the colloidal preparation comprises at least one small-molecule and/or biotechnological active ingredient, wherein, the active ingredient is selected from the group consisting of glucocorticoids, calcineurin inhibitors, Janus kinase inhibitors, antibiotics, and/or a protein in and of itself, a fraction protein, peptide, enzyme, antibody, antibody fragment, RNA and/or DNA molecule, and mixtures thereof, or a molecule which represents a target for one of these active ingredients, and/or the colloidal preparation comprises cosmetic active ingredients and excipients selected from the group consisting of amino acids, urea, glycerol, hyaluronic acid, sugars, sugar-like derivatives, extracts or waxes from plants, or parts derived from animals, and mixtures thereof.

34. The method according to claim 31, wherein the colloidal preparation includes a liquid base, a semi-solid base, or a solid base.

35. A food supplement, an animal feed, a fertilizer, a plant protection product, a soil protection product, a technical additive, a packaging material, a consumer good, a textile, fiber, or a medical product produced from the keratin powder according claim 29.
